(19) United States
(12) Patent Application Publication
(10) Pub. No.: US 2026/0366072 A1
(43) Pub. Date: Nov. 19, 2026

(54) MULTIPLEX METHOD FOR MULTIPLE BIOMARKER DETECTION ON PARTICLES

(71) Applicant: Mercy Bioanalytics, Inc., Waltham, MA (US)

(72) Inventor: Toumy Guettouche, Waltham, MA (US)

(21) Appl. No.: 19/656,471

(22) Filed: Apr. 23, 2026

Related U.S. Application Data
(60) Provisional application No. 63/794,105, filed on Apr. 24, 2025.

(51) Int. Cl. C12Q 1/6869; C12Q 1/25; C12Q 1/6855; C12Q 1/6876

(52) U.S. Cl. CPC C12Q 1/6869; C12Q 1/25; C12Q 1/6855; C12Q 1/6876; C12Y 600/00; C12Q 2600/112; C12Q 2600/16; G01N 2400/00; G01N 2800/60

(57) ABSTRACT

Methods, kits, and systems for detecting cancer, such as lung adenocarcinoma (LUAD), are disclosed. The methods involve analyzing biological particles (e.g., extracellular vesicles or non-vesicular particles) from a sample. Particles are captured via a biomarker associated with the condition. Subsequently, the simultaneous presence of additional biomarkers on the captured particle is detected. Detection of particles displaying the multi-marker co-localization signature is correlated with the presence or state of a condition. Kits and systems for performing the methods are also described.

### TECHNICAL FIELD

The present disclosure relates to methods, kits, and systems for detecting cancer, such as lung cancer, by analyzing co-localized biomarker signatures on biological particles, including extracellular vesicles and non-vesicular particles.

### BACKGROUND

Cancer research has increasingly focused on analyzing biological particles shed by cells into circulation, such as Extracellular Vesicles (EVs) (e.g., exosomes and microvesicles), and various Non-Vesicular Particles (NVPs) (e.g., exomeres, supermeres, lipoproteins, and protein/genomic aggregates). These particles can carry a diverse cargo of proteins, lipids, nucleic acids, and carbohydrates reflective of their cell of origin, including tumor cells. Analyzing biomarkers associated with these particles holds potential for non-invasive cancer detection.

However, achieving high diagnostic accuracy, particularly the specificity needed to distinguish early cancer from benign conditions or healthy states, remains a challenge when relying on the detection of single markers on these particles or bulk analysis of particle populations. There is a clear and unmet need for improved non-invasive diagnostic methods for cancers, e.g., lung cancer, that offer enhanced sensitivity and specificity for early detection, differential diagnosis, and treatment monitoring. Methods capable of detecting specific combinations of multiple biomarkers present simultaneously on individual circulating particles may provide a solution. The present disclosure addresses this need by providing novel methods focused on detecting the co-localization of multiple distinct biomarkers on target particles captured via a first biomarker.

### SUMMARY

The present disclosure provides novel methods, compositions, kits, and systems for the improved detection and diagnosis of cancer, e.g., lung adenocarcinoma (LUAD), through the analysis of particles derived from a subject's sample. Addressing the significant challenges in early and accurate cancer diagnosis, the methods of the present disclosure utilize involve identifying multi-marker signatures on individual particles.

Methods of the present disclosure involve capturing a target population of particles, which may be Extracellular Vesicles (EVs) or Non-Vesicular Particles (NVPs), using a capture agent specific for a first biomarker associated with a condition (e.g., a LUAD-associated protein such as EGFR or CEACAM5). Following isolation, the captured particles are analyzed to detect the simultaneous presence (i.e., co-localization) of at least three additional distinct biomarkers (e.g., a second, third, and fourth biomarker) on the same individual particle. This requirement for detecting a complex signature involving at least four total biomarkers (one capture and at least three detected) aims to confer high specificity to the assay.

The co-localized biomarkers may include various molecular types, such as proteins (e.g., PD-L1), carbohydrates (e.g., Sialyl Lewis X), lipids, or nucleic acids, depending on the particle type (EV or NVP). Detection of co-localization may be accomplished using proximity-dependent assays, such as the Proximity Ligation Assay (PLA), which generates a quantifiable signal (e.g., via qPCR or Next-Generation Sequencing (NGS)) only when the target biomarkers, bound by specific probes, are within close spatial proximity on the same particle. Alternative single-particle analysis techniques such as advanced flow cytometry or super-resolution microscopy may also be employed.

By analyzing these specific multi-marker co-localization signatures on individual particles, the methods described herein offer the potential for enhanced sensitivity and specificity compared to conventional diagnostic approaches for cancers, e.g., LUAD, such as imaging or single-biomarker tests. The methods of the present disclosure may be used for various clinical purposes, including screening of high-risk populations (e.g., smokers), differential diagnosis of cancer, early detection of cancer e.g., LUAD, and monitoring therapeutic response or disease recurrence.

The disclosure further encompasses kits containing the necessary reagents (e.g., capture agents, detection probes, enzymes) and systems (e.g., automated or microfluidic platforms) designed to perform the described particle capture and co-localization detection methods.

### BRIEF DESCRIPTION OF DRAWINGS

FIG. 1 shows a schematic representation of a captured extracellular vesicle (EV) with three distinct biomarkers. The EV is surrounded by three different antibodies, each used to detect one of the three distinct biomarkers on the surface of the EV.

### DETAILED DESCRIPTION

The present disclosure provides methods, compositions, kits, and systems for detecting conditions, such as Lung Adenocarcinoma (LUAD), in a subject by analyzing specific combinations of biomarkers associated with individual biological particles obtained from a biological sample. A biomarker refers to a distinct molecular indicator associated with a particular biological state or condition and may include proteins, lipids, carbohydrates, nucleic acids, or modifications thereof. There is a need for improved diagnostic tools for lung cancer, e.g., for early detection in high-risk populations (e.g., smokers) as current methods often lack sufficient sensitivity or specificity. The methods of the present disclosure address this need by providing a novel approach involving the analysis of multiple co-localized biomarkers on specific particle populations. This approach offers improved diagnostic accuracy compared to conventional methods. The method involves capturing target particles based on a first biomarker and subsequently detecting the co-localization of at least three additional biomarkers on the same captured particle, resulting in analysis of at least four total biomarkers, using, for example, proximity-dependent assays such as the Proximity Ligation Assay (PLA). The methods described herein achieve high diagnostic accuracy suitable for early LUAD detection or monitoring.

Methods of the present disclosure may involve liquid biopsy techniques. Liquid biopsy is a minimally invasive technology that involves the analysis of biomarkers in bodily fluids (e.g., blood, urine, saliva) to detect, diagnose, and monitor diseases such as cancer. Liquid biopsy techniques avoid the need for invasive tissue biopsies by analyzing circulating analytes like ctDNA, CTCs, and various types of biological particles shed from tumors or affected tissues.

### Biological Particles

Biological fluids contain a complex mixture of particles derived from cells. The methods described herein are applicable to analyzing biomarkers on various types of such particles, including both Extracellular Vesicles (EVs) and Non-Vesicular Particles (NVPs).

Extracellular Vesicles (EVs) are vesicular particles enclosed by a lipid bilayer. EVs may be released from cells. EVs play roles in intercellular communication and waste removal, and their cargo (e.g., proteins, lipids, nucleic acids) may reflect the state of their cell of origin. Major types of EVs relevant to diagnostics include exosomes, microvesicles, apoptotic bodies, and oncosomes.

Exosomes are small EVs, about 30-150 nm, originating from the endosomal pathway. Microvesicles, or ectosomes, are larger EVs, about 100-1000 nm, budding directly from the plasma membrane. Apoptotic bodies are vesicles released during programmed cell death (i.e., apoptosis). Oncosomes are large EVs shed by cancer cells.

Non-Vesicular Particles (NVPs) are subcellular or supramolecular entities not enclosed by a continuous lipid bilayer. Examples include lipoproteins, protein aggregates, genomic aggregates, ribonucleoprotein complexes, exomeres, and supermeres. Lipoproteins, such as LDL, HDL, and VLDL, carry lipids and comprise lipids and proteins. Protein aggregates may include disease-associated aggregates or aggregated oncoproteins. Genomic aggregates are complexes involving DNA fragments. Ribonucleoprotein complexes are assemblies of RNA and proteins. Exomeres and supermeres are specific classes of nanoparticles lacking a lipid bilayer. Exomeres and supermeres may carry proteins, metabolites, and nucleic acids. Exomeres/Supermeres may be distinct populations of nanoparticles lacking a lipid bilayer found in bodily fluids, smaller than exosomes. Exomeres, about 35 nm, may contain metabolic enzymes and proteins related to hypoxia and glycolysis. Supermeres, larger than 50 nm, may overlap in size with small EVs and carry proteins involved in coagulation and immunity, along with lipids and nucleic acids.

Both EVs and NVPs can originate from various cells, including lung adenocarcinoma cells, via secretion, shedding, or release during cellular processes. Their composition can change in response to disease states like cancer, making them useful for the co-localization biomarker analysis described herein. A biomarker is considered “associated with” a particle if it is physically part of it, bound to its surface, contained within its structure, or located within its lumen or membrane (e.g., for EVs).

### Isolated Particles

Following isolation, analysis of the particle population, e.g., to determine whether EV or NVP, may be performed. Techniques such as Nanoparticle Tracking Analysis (NTA) or Dynamic Light Scattering (DLS) may provide information on size distribution. Electron microscopy (e.g., TEM, SEM) may provide morphological information (e.g., distinguishing vesicular from non-vesicular structures). Proteomic analysis (e.g., mass spectrometry) or Western blotting may confirm the presence of biomarkers (e.g., EV markers including tetraspanins CD9/CD63/CD81, or specific NVP components) and identify other associated molecules.

### Sample Collection and Preparation

The methods utilize a sample obtained from a subject. Suitable samples containing relevant particles include, but are not limited to, whole blood, plasma, serum, urine, saliva, cerebrospinal fluid (CSF), ascites fluid, pleural effusion, lymph, tears, and sweat. Samples are collected using standard clinical procedures (e.g., venipuncture for blood). Plasma or serum is used for analyzing circulating particles relevant to lung cancer and is isolated by centrifugation to remove blood cells. Urine may be used for detecting bladder or kidney cancer markers, while saliva may be used for oral or head and neck cancer detection, and specific standardized collection protocols for those sample types are used. Additional centrifugation or filtration to remove large debris, may be performed. Samples may be analyzed fresh or stored, e.g., frozen (e.g., at −80 °C), ensuring particle and biomarker integrity until analysis. Other variables taken into consideration in sample collection and preparation include sample collection tube type, time between collection and processing, centrifugation protocols, and storage conditions (e.g., temperature, freeze-thaw cycles).

### Isolation of Target Particles

The methods of the present disclosure involve isolating a target population of particles (e.g., EVs and/or NVPs) from the biological sample based on a first biomarker associated with these particles (e.g., particles linked to LUAD). This biomarker serves as an anchor for capture.

Isolation uses a capture agent specific for this first biomarker. Capture agents include antibodies, antibody fragments, aptamers, or other specific ligands. The first biomarker may be a protein (e.g., EGFR, CEACAM5) or a carbohydrate moiety (e.g., SialylTn, Sialyl Lewis X).

Aptamers are short, single-stranded nucleic acid sequences (DNA or RNA) that can fold into complex three-dimensional structures capable of binding to proteins, small molecules, or cells. They are generated through an in vitro selection process (SELEX).

Immunoaffinity capture is a common technique, often using the capture agent immobilized on a solid support (e.g., magnetic beads, plates, microfluidic channels). For example, particles may be isolated from plasma using a first antibody immobilized on magnetic beads. The sample is contacted with the support, target particles bind, and unbound material is washed away, resulting in an isolated population of the target particles bound to the support. Other methods including size-exclusion chromatography, density gradient centrifugation, or precipitation may be employed as preliminary or alternative isolation steps.

Immunoaffinity Capture relies on the highly specific binding interaction between an antibody, or other ligand, and its target antigen (e.g., the biomarker). By immobilizing the antibody/ligand, it can be used to selectively “pull down” or capture molecules or particles bearing the target antigen from a sample.

### Detection of Co-Localized Biomarkers

Following isolation, the captured particles are analyzed to detect individual particles that simultaneously display at least three additional biomarkers (e.g., a second, third, and fourth distinct biomarker) in a co-localized manner. This multi-marker co-localization detection involving analysis of at least four total biomarkers (one capture and at least three detected) is central to achieving high specificity for conditions such as LUAD.

These additional biomarkers are selected for their combined diagnostic value for the target condition. They may be proteins (e.g., PD-L1, CEACAMs), carbohydrates (e.g., Sialyl Lewis X), lipids, or nucleic acids. These detected biomarkers may be of the same or different molecular types as each other and the capture biomarker. For instance, the capture biomarker and all the co-localized biomarkers detected may be proteins. In another embodiment, capture might be via a protein, while detection involves co-localization of another protein and a carbohydrate. In a certain embodiment, an additional biomarker is a carbohydrate/glycan structure, or a lipid/glycolipid such as phosphatidylserine or a ganglioside. The biomarker is detected via a method involving PLA. In another embodiment, an additional biomarker is a protein. The biomarker is detected via a method involving PLA. In another embodiment, the additional biomarker is an RNA molecule such as mRNA, miRNA, lncRNA, etc. The biomarker is detected via nucleic acid amplification methods (e.g., qRT-PCR).

Methods of the present disclosure for detecting co-localization may involve a Proximity Ligation Assay (PLA). This method involves the use of distinct probes specific for each of the multiple additional biomarkers being detected (i.e., at least three distinct probes corresponding to the three additional biomarkers). Each probe comprises a target binding moiety (e.g., an antibody or aptamer) linked to an oligonucleotide. The oligonucleotide may comprise a single-stranded portion. The oligonucleotide may comprise a sticky end. The method further involves contacting the isolated particles with the probes. If a particle displays the target biomarkers in close proximity, the corresponding probes will bind nearby. The proximity of the bound probes allows their single stranded portions of the attached oligonucleotides to hybridize. A nucleic acid ligase enzyme joins these hybridized single stranded portions, creating a ligated nucleic acid template. The method further involves detecting and quantifying the ligated template using methods such qPCR, dPCR, or DNA Sequencing (e.g., Next-Generation Sequencing, NGS). The resulting signal reflects the number of particles positive for the specific multi-marker co-localization signature.

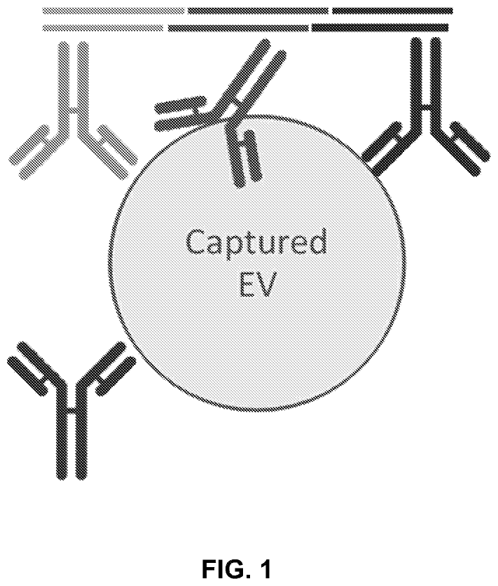

FIG. 1 schematic representation of a method involving detection of extracellular vesicles (EVs) displaying up to four distinct biomarkers. The method involves using three distinct biomarkers that must be colocalized to generate a signal. This method allows for the construction of more specific biomarker combinations with lower background signals in healthy control samples. The schematic shows a captured EV surrounded by three different antibodies, each used to bind one of the three distinct biomarkers on the EV. The use of multiple biomarkers enhances the specificity and sensitivity of biomarker detection.

Alternative methods capable of detecting multiple markers on single particles may also be used. These include advanced multi-parameter flow cytometry, utilizing instruments and protocols adapted for small particle analysis (e.g., EV-flow cytometry), or high-resolution microscopy techniques (e.g., super-resolution fluorescence microscopy such as Stimulated Emission Depletion (STED) microscopy or platforms including the ONI Nanoimager), or electron microscopy with immunogold labeling. Other sensitive detection platforms may be used for detecting co-localized biomarkers on isolated particles. For instance, single-molecule detection techniques like Single Molecule Array (SIMOA) may be used to detect the co-capture of multiple detection probes on the same particle. Surface Plasmon Resonance (SPR) may be used to monitor sequential or simultaneous binding events on captured particles indicative of co-localization. Advanced mass spectrometry combined with chemical cross-linking may identify co-localized proteins. Spectroscopic methods such as Raman spectroscopy, enhanced with Surface-Enhanced Raman Scattering (SERS) tags conjugated to probes, may also provide molecular information related to co-localization.

Examples of carbohydrates and lipids that may be used in a biomarker signature include, but are not limited to Tn antigen, SialylTn (sTn) antigen, Thomsen-Friedenreich (T, TF) antigen, Lewis Y (also known as CD174) antigen, Lewis B antigen, Sialyl Lewis X (sLex) (also known as Sialyl SSEA-1 (SLX)) antigen, SSEA-1 (also known as Lewis X), beta1,6-branching, bisecting GlcNAc in a beta1,4-linkage, core fucosylation, Sialyl-T antigens (sT), Sialyl Lewis c antigen, Globo H, SSEA-3 (Gb5), SSEA-4 (sialy-Gb5), Gb3 (Globotriaose, CD77), Disialosyl-galactosylgloboside (DSGG), GalNAcDSLc4, Fucosyl GM1, GD1alpha, GD1a ganglioside, GD2 ganglioside, GD3 ganglioside, GM2 ganglioside, Lc3 ceramide, nLc4 ceramide, 9-O-Ac-GD2 ganglioside, 9-O-Ac-GD3 (CDw60) ganglioside, 9-O-Ac-GT3 ganglioside, Forssman antigen, Disialyl Lewis a antigen, Sialylparagloboside (SPG), Polysialic acid (PSA) linked to NCAM, Sialyl Lewis A antigen (also known as CA19-9), CanAg (glycoform of MUC1), Lewis Y/B antigen, Sialyltetraosyl carbohydrate, NeuGcGM3, GM3 (N-glycolylneuraminic acid (NeuGc, NGNA)-gangliosides GM3), phosphatidylserine, and combinations thereof.

Examples of proteins that may be used in a biomarker signature include, but are not limited to ALDH18A1, AP1M2, APOO, ARFGEF3, B3GNT3, BMPR1B, CADM4, CANT1, CD24, CDH1, CDH17, CDH2, CDH3, CEACAM5, CEACAM6, CLDN3, CLDN4, CLGN, CLN5, CYP2S1, DSG2, ELAPOR1, ENPP5, EPCAM, EPHB2, FAM241B, FERMT1, FOLR1, FZD2, GALNT14, GALNT6, GJB1, GNG4, GNPNAT1, GOLM1, GPR160, GPRIN1, GRHL2, HACD3, HS6ST2, IGSF3, ILDR1, KDELR3, KPNA2, KRTCAP3, LAMB3, LAMC2, LAPTM4B, LARGE2, LMNB1, LRRN1, LSR, MAL2, MARCKSL1, MARVELD2, MET, MUC1, MUC2, MUC4, MUC5AC, MUC13, NPTXR, NUP210, PARD6B, PMEPA1, PODXL2, PRAF2, PRSS8, RAB25, RAC3, RACGAP1, RAP2B, RCC2, RNF128, RNF43, RPN1, RPN2, SERINC2, SHISA2, SLC35A2, SLC39A6, SLC44A4, SLC4A4, SMIM22, SMPDL3B, SYAP1, SYT13, TMEM132A, TMEM238, TMEM9, TSPAN13, ULBP2, UNC13B, VTCN1, ABCA13, ADAM23, CYP4F11, HAS3, TMPRSS4, UGT1A6, PIGT, TOMM34, ACSL4, GPC3, ROBO1, SLC22A9, SLC38A3, TFR2, TM4SF4, TMPRSS6, ANXA13, CHST4, GAL3ST1, SNAP25, TMEM156, CLDN18, EPPK1, MUC13, OCLN, CFTR, GCNT3, ITGB6, ITGB6, LAD1, MSLN, TESC, LYPD6B, S100P, TMEM51, TNFRSF21, UPK1B, UPK2, ABCC4, FOLH1, RAB3B, STEAP2, TMPRSS2, TSPAN1, AP1S3, DSC2, DSG3, TMPRSS11D, KCNS1, LY6K, MUC4, SYNGR3, CELSR1, COX6C, ESR1, MUC1, ABCC11, ERBB2, SLC9A3R1, PROM1, PTK7, CDK4, DLK1, LMNB2, PCDH7, TMEM108, TYMS, SDC1, SLC34A2, BCAM, MUC16, ADAM17, ADAM28, ADAM8, ALCAM, AMHR2, AXL, BAG3, BSG, CCL2, CCL8, CCN1, CCN2, CCR5, CD274, CD38, CD44, CD47, CDH11, CETN1, CLDN1, CLEC2D, CLU, CSPG4, DKK1, DLL4, EGFR, ENPP3, EPHA10, ERBB3, FAP, FGF1, FGFR4, FLNA, FLNB, FLT4, FZD7, GFRA1, GM3, GPA33, GPC1, GPNMB, GUCY2C, HGF, ICAM1, IGF1R, IL1A, IL1RAP, IL6, ITGA6, ITGAV, KDR, KLK3, KLKB1, KRT8, LAG3, LGR5, LPR6, LY6E, MCAM, MDM2, MELTF, MERTK, MST1R, MUC17, MUC5AC, MUCL1, NOTCH2, NOTCH3, NRP1, NT5E, PI4K2A, PLAC1, PLAUR, PLVAP, PPP1R3A, PRLR, PSCA, PVR, RET, S1PR1, SLC3A2, SLC7A11, SLC7A5, SPINK1, STAT3, STEAP1, TACSTD2, TF, TFRC, TGFBR2, TIGIT, TNC, TNFRSF10A, TNFRSF10B, TNFRSF12A, TNFRSF4, TNFSF11, TNFSF18, TPBG, VANGL2, VEGFA, VEGFC, and combinations thereof.

Biomarkers may include lipids such as phospholipids and glycolipids associated with the particle, carbohydrates e.g., glycans on proteins or lipids, nucleic acids including various forms of intravesicular RNA (e.g., mRNA, microRNA, long noncoding RNA, circular RNA, etc.), or modifications thereof. The methods of the present disclosure involve capturing target particles based on a first biomarker and subsequently detecting the co-localization of multiple additional biomarkers on the same captured particle, using, for example, proximity-dependent assays like the Proximity Ligation Assay (PLA) for detecting protein biomarkers or nucleic acid amplification methods like qRT-PCR for detecting RNA markers.

### Correlation With Condition

Methods of the present disclosure may also involve correlating the detected co-localization signal with the presence, absence, or state of the condition (e.g., lung cancer) in the subject. The quantitative output from the detection step (e.g., qPCR Ct value, number of positive events from flow cytometry) is compared against reference values. This may involve comparison to a predetermined diagnostic threshold, established through clinical studies on patient cohorts with known disease status. Signals exceeding the threshold may indicate the presence of the condition. Alternatively, signals may be compared to those from control groups (e.g., healthy individuals, individuals with benign conditions) to assess differences.

High diagnostic specificity relies on the detection of multi-marker co-localization. Methods of the present disclosure include using appropriate controls. In certain embodiments, the methods include processing samples from confirmed healthy individuals and subjects with relevant benign conditions; using isotype controls or irrelevant binding agents in parallel with specific capture agents and probes; omitting key enzymes (e.g., ligase in PLA) to assess background signal; and including positive and negative control materials with known particle/biomarker compositions.

The quantitative results provided by the methods of the present disclosure allow for monitoring of disease progression or treatment response, for instance, by comparing results from longitudinal samples taken from a subject undergoing therapy. The methods may also be applied for screening asymptomatic subjects considered high-risk for a specific cancer type (e.g., based on genetic predisposition or family history).

In embodiments where multiple distinct co-localization signatures are measured simultaneously across different biomarker combinations (i.e., multiplexing), data analysis may involve normalization, statistical modeling, or machine learning algorithms (e.g., logistic regression, support vector machines, decision trees) trained on clinical datasets to integrate the multiplex data into a single diagnostic score or classification.

### Biomarker Selection and Validation Strategy

The selection of the set of biomarkers (e.g., one capture, plus at least three co-localized detection markers) plays an important role in the methods of the present disclosure. Candidate biomarkers may be identified through discovery via comparative multi-omics (e.g., proteomics, glycomics, etc.) of particles from diseased vs. control subjects. Public databases and literature searches may also inform candidate biomarker selection. For example, in Lung Adenocarcinoma (LUAD), the methods involve capturing particles via an antibody to EGFR (Epidermal Growth Factor Receptor) and detecting co-localization of CEACAM5, PD-L1, and a specific glycoform of CK7 (Cytokeratin 7). In another embodiment, the method involves capturing via CEACAM5 and detecting co-localization of EGFR, Napsin-A associated protein/epitope, and PD-L1.

The specific combination of capture and the at least three detection markers may be optimized for sensitivity and specificity for LUAD, and not for other lung cancer subtypes (e.g., squamous cell carcinoma) or benign lung conditions. Similar strategies may be applied to other cancers by selecting appropriate biomarkers. For example, MUC1, CEACAMs, FOLR1, STn, or SLeX may be relevant for other adenocarcinomas like ovarian or colorectal cancer. Candidate biomarkers and their combinations may be validated using techniques such as Western blotting, ELISA, mass spectrometry, or preliminary co-localization assays on well-characterized clinical cohorts to confirm their association with the target condition, their presence on the target particles (EVs or NVPs), and their ability to provide diagnostic information when assessed in combination.

### Conditions for Detection

There is a need for better early detection and characterization tools for cancer. The methods are broadly applicable to cancers (e.g., lung, ovarian, pancreatic, breast, colorectal, etc.) and other diseases where specific multi-marker particle signatures exist.

Lung cancer is the leading cause of cancer death globally. Early detection significantly improves survival, but low-dose CT screening, while beneficial for high-risk individuals, has limitations regarding false positives and detection of indolent or aggressive disease. The methods of the present disclosure may involve non-invasive liquid biopsy tests that accurately detect early-stage LUAD and/or differentiate subtypes.

### Clinical Use

The methods described herein may have clinical uses that relate to lung cancer and other diseases. The methods may be used for screening in high-risk individuals (e.g., heavy smokers) alongside or as an adjunct to imaging e.g., low-dose CT, improving specificity. The methods may be used to identify LUAD at stages I/II when curative treatment is more likely. The methods may provide information on biomarker expression (e.g., PD-L1, EGFR status via protein co-localization) relevant to targeted therapy or immunotherapy selection. The methods may be used for tracking and monitoring treatment response or detecting minimal residual disease/recurrence earlier than imaging. The method may be optimized based on the specific intended use.

### Kits and Systems

Kits would contain reagents specific for certain applications, such as a LUAD detection. In a certain embodiment, the kit comprises a capture agent (e.g., anti-EGFR antibody on beads); at least three sets of detection probes (e.g., PLA probes for CEACAM5, PD-L1, CK7-glycoform); enzymes, buffer solutions, controls, and instructions. Systems may be used to automate the process (e.g., microfluidic systems/platforms) integrating the steps involving isolation of particles, performance of reactions, and detection of co-localized biomarkers.

Variations in specific biomarkers, the number of co-localized biomarkers detected (e.g., at least three are detected in addition to the capture marker), particle types (EVs or NVPs), reagents, detection methods, sample types, and target conditions are all contemplated within the scope of the invention described herein.

### Incorporation by Reference

References and citations to other documents, such as patents, patent applications, patent publications, journals, books, papers, web contents, publicly accessible databases, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of detecting a condition, the method comprising:
capturing a particle from a sample with a capture agent specific for a first biomarker of the condition;
detecting co-localized second, third, and fourth biomarkers of the condition on the captured particle to determine the presence of the condition.

2. The method of claim 1, wherein the condition is cancer.

3. The method of claim 2, wherein the cancer is ovarian cancer.

4. The method of claim 1, wherein the sample is obtained from whole blood, plasma, serum, urine, saliva, or cerebrospinal fluid, 5. The method of claim 2, wherein the cancer is selected from ovarian cancer, pancreatic cancer, lung cancer, breast cancer, and colorectal cancer.

6. The method of claim 1, wherein the first, second, third, and fourth biomarkers are all proteins.

7. The method of claim 1, wherein the first biomarker is a protein and at least one of the second, third, or fourth biomarkers is a carbohydrate.

8. The method of claim 1, wherein the first biomarker is a carbohydrate.

9. The method of claim 1, wherein the carbohydrate is a SialylTn or Sialyl Lewis X.

10. The method of claim 8, wherein the second biomarker is a carbohydrate.

11. The method of claim 1, wherein the first and second biomarkers are a protein.

12. The method of claim 1, wherein the particle is an extracellular vesicle.

13. The method of claim 1, wherein the particle is selected from the group consisting of exosome, microvesicle, apoptotic body, and oncosome.

14. The method of claim 1, wherein the particle is selected from the group consisting of exomere, supermere, proteomic aggregate, and genomic aggregate.

15. The method of claim 1, wherein the second, third, and fourth colocalized biomarkers are each selected from the group consisting of a protein, a nucleic acid, and a carbohydrate moiety.

16. The method of claim 1, wherein the particle is not enclosed by a lipid bilayer and/or non-vesicular.

17. The method of claim 1, further comprising
contacting the captured particle with a first, second, and third probe, each probe comprising:
(i) a binder specific to one of the colocalized biomarkers, and
(ii) an oligonucleotide linked to the binder
binding each probe to its respective colocalized biomarker;
attaching the oligonucleotides to form a product; and
detecting the product.

18. The method of claim 1, wherein the capture agent is specific to an epitope of the biomarker of the condition.

19. The method of claim 18, wherein the colocalized biomarkers include a different epitope of the biomarker.

20. The method of claim 14, wherein the oligonucleotides comprise a single-stranded portion.

21. The method of claim 17, wherein the attaching step comprises ligating the oligonucleotides using a ligase.

22. The method of claim 21, wherein the detecting step comprises Next-Generation Sequencing.
